(19) United States
(12) Patent Application Publication
(10) Pub. No.: US 2016/0128919 A1
(43) Pub. Date: May 12, 2016

(54) NON-INVASIVE AND LONG LASTING SKIN DARKENING DEVICES, COMPOSITIONS, AND METHODS OF USE FOR BODY CONTOURING

(71) Applicant: Dagmar Bjork SVEINE, San Diego, CA (US)

(72) Inventor: Dagmar Bjork SVEINE, San Diego, CA (US)

(21) Appl. No.: 14/934,989

(22) Filed: Nov. 6, 2015

Related U.S. Application Data
(60) Provisional application No. 62/076,326, filed on Nov. 6, 2014.

(51) Int. Cl. A61K 8/35; A45D 40/26; A45D 40/20; A61Q 1/02; A45D 40/30

(52) U.S. Cl. CPC A61K 8/35; A61Q 1/02; A45D 40/30; A45D 40/20; A45D 40/262

(57) ABSTRACT

The disclosure relates to devices, compositions, and methods to create long lasting cosmetic contouring on a human body. Cosmetic contouring aesthetically enhances visual appearance by creating the appearance of depth to highlight certain features and/or minimize other features. Cosmetic contouring can be applied to the entire body, however, it is most frequently used on the face. In one embodiment the disclosure relates to using skin darkening compounds such as dyes or sunless tanning agents to create a long lasting cosmetic contouring effect.

### CROSS-REFERENCE TO RELATED APPLICATIONS

This application claims the benefit of U.S. provisional application No. 62/076,326, filed on Nov. 6, 2014 and entitled, “Non-invasive and long lasting, skin darkening devices, compositions, and methods of use for body contouring.” The entire contents of the provisional application are incorporated by reference herein.

### TECHNICAL FIELD

The disclosure relates to devices, compositions, and methods to create long lasting cosmetic contouring on a human body. Cosmetic contouring aesthetically enhances visual appearance by creating the appearance of depth to highlight certain features and/or minimize other features. Cosmetic contouring can be applied to the entire body, however, it is most frequently used on the face. In one embodiment the disclosure relates to using skin darkening compounds such as dyes or sunless tanning agents to create a long lasting cosmetic contouring effect.

### BACKGROUND

In pursuit of beauty, Americans spent more than $12 billion on surgical and non-surgical cosmetic procedures and approximately $56.63 billion on cosmetics in 2012. (See The American Society for Aesthetic Plastic Surgery 17th annual multi-specialty statistical data and Statista report for cosmetics and personal care.) Changing one's appearance can be achieved on a permanent basis through the use of surgical procedures (i.e. implants, rhinoplasty, liposuction, etc), on a long-term basis through the use of non-surgical procedures (i.e. Botulinum toxin, chemical peels, injectable fillers, etc.) or on a temporary basis through the use of makeup. While surgical and non-surgical procedures provide permanent or long-term results, the procedures carry a risk of complications, unnatural appearance and scarring. Additionally, surgical and non-surgical procedures are expensive and should only be performed by competent certified personnel. Makeup, on the other hand, can be inexpensive and involves less risk health-wise. However, the effect of makeup only lasts until the makeup is washed off or has worn off.

Cosmetic contouring is the art of using makeup to diminish or enhance features of the body. In general, makeup that is darker than the natural color of the skin is used to create shadowed regions and makeup that is lighter than the natural color of the skin is used to create highlighted regions. For example, applying darker shades under the cheekbone gives the appearance of higher cheekbones, a thinner face, and smaller cheeks. Applying darker shades on the jaw can make a round face appear more elongated. Applying darker shades to the sides of the nose creates the appearance of a thinner and longer nose. Similarly, the use of highlighting and shadow can be used to enhance the appearance of muscles or cleavage.

A variety of makeup products are available to affect cosmetic contouring. One such example are bronzers. Bronzers are a temporary sunless tanning or bronzing option. These come in powders, sprays, mousse, gels, lotions and moisturizers. Once applied, they create a tan that can easily be removed with soap and water. Bronzers are typically used to add a “sun-kissed” look to a pale or sallow skin tone. It is generally applied by sweeping colors on areas the sun naturally touches such as the “apple” of the cheek, forehead, nose, and chin.

While the use of makeup to alter one's appearance is an affordable option for many people, it can be time consuming to apply on a daily basis. Accordingly, it would be useful to have a product that would reduce the amount of time it takes to create a cosmetic contoured look.

### SUMMARY

The following simplified summary provides a basic understanding of some aspects of the claimed subject matter. This summary is not an extensive overview, and is not intended to identify key/critical elements or to delineate the scope of the claimed subject matter. Its purpose is to present some concepts in a simplified form as a prelude to the more detailed description that is presented below

In one embodiment is a device for applying a skin darkening agent in a discrete location to accentuate body contours, such as facial contours or muscle contours. The devices include a skin darkening agent and an applicator to use to apply the skin darkening agent to the skin. In one embodiment the applicator is a patch. In another embodiment, the patch is pre-shaped to contours parts of a face. In another embodiment, the patch is pre-shaped to contour a cheek. In another embodiment, the patch is pre-shaped to contour a nose. In another embodiment, the patch is pre-shaped to contour a forehead. In another embodiment, the patch is pre-shaped to contour a jawline. In another embodiment, the patch is pre-shaped to contour parts of a neck. In another embodiment, the patch is pre-shaped to contour parts of a chest. In another embodiment, the patch is pre-shaped to contour parts of an arm. In another embodiment, the patch is pre-shaped to contour parts of a leg. In another embodiment, the patch is pre-shaped to contour parts of an abdomen. In another embodiment, any of the above mentioned patches may include an adhesive layer. In yet another embodiment, the applicator is a stick like device. In another embodiment, the applicator is sponge. In another embodiment, the applicator is a brush. In another embodiment, the brush has a handle that may be hollow. In another embodiment the hollow handle may include a mechanical means for extruding a composition located inside the hollow handle. In yet another embodiment, the device is a compact that includes either a brush, sponge, or pad.

In one embodiment, any of the above discussed devices contain a skin darkening agent. In another embodiment, the skin darkening agent may be dihydroxyacetone, erythrulose, henna, glucose tyrosinate, acetyl tyrosine, brazilin, caffine, coffee extracts, isobutyl methyl xanthine, methyl xanthine, tea extracts, theophylline, isatin, alloxan, ninhydrin, glyceraldehyde, mesotartaric aldehyde, glutaraldehyde, pyrazoline-derivatives of 4,5-diones, derivatives of 4,4-dihydroxypyrazolin-5-ones, or walnut extract, cacao extract, spice extract, fruit extract, vegetable extract, or berry extract. In another embodiment, the skin darkening agent may be a mixture of at least two skin darkening agents. In another embodiment, the skin darkening agent may be a mixture of at least three skin darkening agents. In another embodiment, the skin darkening agent is dihydroxyacetone. In another embodiment, the skin darkening agent is erythrulose. In another embodiment, the skin darkening agent is henna. In another embodiment, the skin darkening agent may be a spice extract. The spice extract may be from saffron, curry, turmeric, cinnamon, cloves, allspice, nutmeg, paprika, rosemary, vanilla, or cumin. In another embodiment, the skin darkening agent is a fruit extract. The fruit extract may be from grapes or tomatoes. In another embodiment, the skin darkening agent may be a vegetable extract. The vegetable extract may be from carrots, beets, or cabbage. In another embodiment, the skin darkening agent may be a berry extract. The berry extract may be from strawberries, blueberries, blackberries, mulberries, or raspberries.

In one embodiment, the above described skin darkening agents are applied to the skin to creating a non-permanent long-term darkened regions on the skin. The darken regions are placed to enhance or mimic the natural contours of the face or muscles. In another embodiment, the skin darkening agents are applied to areas on a face. In another embodiment, the skin darkening agents are applied to areas on a neck. In another embodiment, the skin darkening agents are applied to areas on a chest. In another embodiment, the skin darkening agents are applied to areas on an abdomen. In another embodiment, the skin darkening agents are applied to areas on an arm. In another embodiment, the skin darkening agents are applied to areas on a leg. In another embodiment, the skin darkening agents are applied to areas on a shoulder. In another embodiment, the skin darkening agents are applied to areas on a back. In another embodiment, the skin darkening agents are applied to areas on a pelvis.

In one embodiment, the above described skin darkening agents are applied to the skin creating long-lasting contour facial makeup. The skin darkening agents are applied to areas where a darker foundation would be applied for contour makeup. In another embodiment, the skin darkening agents are applied to either side of a nose. In another embodiment, the skin darkening agents are applied under the cheek bone. In another embodiment, the skin darkening agents are applied along the jaw. In another embodiment, the skin darkening agents are applied along the hairline. In another embodiment, the skin darkening agents are applied along the chin.

In one embodiment, a person may use a computer system to assist in the placement of contour makeup. The computer system could obtain an image of the face; analyze the face shape; and provide advice on the placement of highlighting makeup and shadowing makeup on the image of the face. In another embodiment, the computer system could also include a camera to capture the image of the face. In another embodiment, the computer system could include a printer that will print patch shapes that are specific for the imaged face. In another embodiment, the computer system could be an app on an electronic device. In another embodiment, the aforementioned app could incorporate the use of the camera on the electronic device to provide the image of the face.

In one embodiment is a topical compound containing a skin darkening agent in a carrier. Exemplary skin darkening agents include dihydroxyacetone, erythrulose, henna, glucose tyrosinate, acetyl tyrosine, brazilin, caffine, coffee extracts, isobutyl methyl xanthine, methyl xanthine, tea extracts, theophylline, isatin, alloxan, ninhydrin, glyceraldehyde, mesotartaric aldehyde, glutaraldehyde, pyrazoline-derivatives of 4,5-diones, derivatives of 4,4-dihydroxypyrazolin-5-ones, walnut extract, cacao extract, spice extract, fruit extract, vegetable extract, and berry extract. Alternatively, the topical compound contains at least two skin darkening agents. In another alternative, the topical compound contains at least three skin darkening agents. In another embodiment, the skin darkening agent can be dihydroxyacetone. In another embodiment, the skin darkening agent can be erythrulose. In another embodiment, the skin darkening agent can be henna. In another embodiment, the skin darkening agent can be a spice extract. In another embodiment, the skin darkening agent can be a fruit extract. In another embodiment, the skin darkening agent can be vegetable extract. In another embodiment, the skin darkening agent can be a berry extract. In another embodiment, the topical compound may also include a chelating agent. In another embodiment, the topical compound may also include an anti-irritant agent. In another embodiment, the topical compound may also include a skin penetration enhancer. In another embodiment, the topical compound may also include a thickener. In another embodiment, the topical compound may also include a skin darkening accelerant.

### BRIEF DESCRIPTION OF THE DRAWINGS

FIG. 1 shows exemplary locations for applying highlighting makeup and shadowing makeup for facial contouring. Regions marked with an “H” are areas where highlighting makeup are applied and regions marked with an “S” are areas where shadowing makeup are applied.

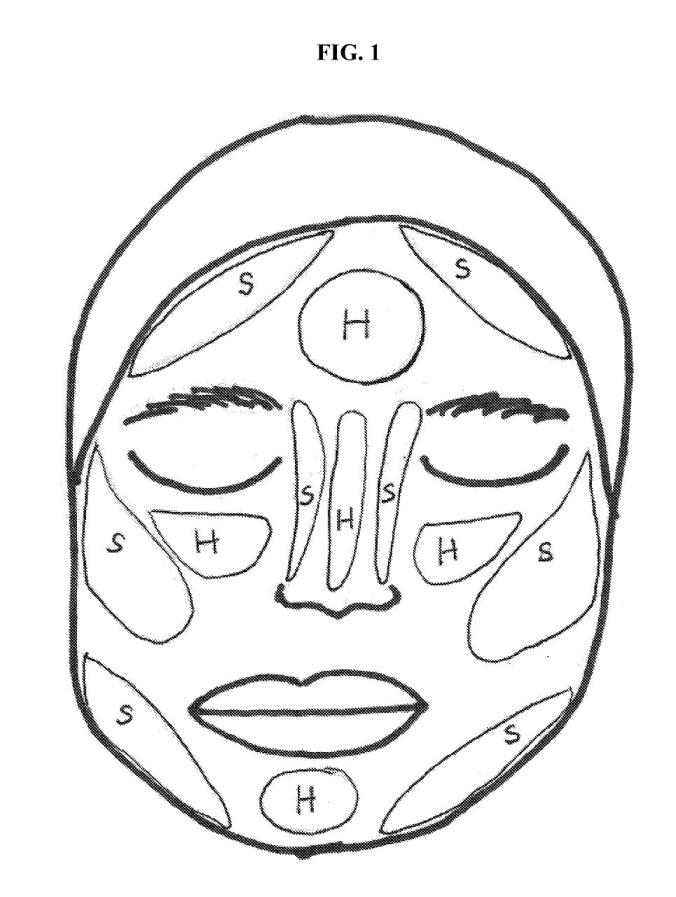

FIG. 2 shows exemplary shapes for pre-shaped patches for facial contouring. The patches are located along the hairline, on the sides of the nose, under the cheekbone, and along the jaw.

FIG. 3 shows an exemplary pre-shaped patch and an exemplary cross-section of a pre-shaped patch.

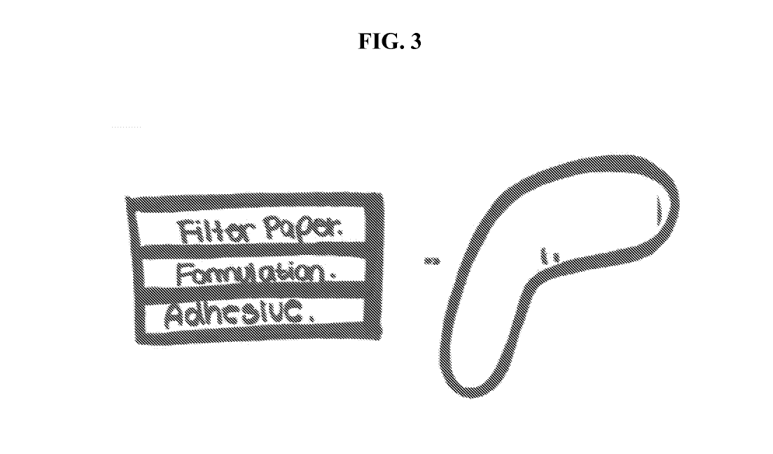

### DESCRIPTION

Contour makeup is an affordable alternative for visually changing one's appearance. Contour makeup is a multistep process that involves (1) applying foundation, (2) applying foundation that is lighter than one's natural skin tone to specific areas of the body to create highlighted areas, (3) applying foundation that is darker than one's natural skin tone to specific areas of the body to create shadow areas, (4) blending the lighter and darker foundations, (5) apply a finishing powder to set the makeup. After applying the contour makeup to the face, additional makeup such as blush, eye shadow, and mascara are then applied. The disclosure set out herein is directed to long-term skin darkening products that when used, will reduce the amount of time it takes to apply contour makeup.

### DEFINITIONS

In the description that follows, a number of terms are extensively utilized. The following non-limiting definitions provide a clear and consistent understanding of the specification and claims, including the exemplary scope to be given such terms.

When the terms “one,” “a,” or “an” are used in this disclosure, they mean “at least one” or “one or more,” unless otherwise indicated.

The terms “invention” or “present invention” as used herein are intended to be non-limiting and are not intended to refer to any single embodiment of the particular invention but encompasses all possible embodiments as described in the specification and the claims and logical extensions thereof.

The term “skin darkening agent” as used herein refers to any compound that can temporarily darken human skin even after removal of the agent. The darkening may be due to staining the skin or alternatively, may be due to stimulation of melanin or other pigment production. Non-limiting examples include dihydroxyacetone, erythrulose, and henna.

The term “sunless tanning agent” as used herein refers to any compound that produces a tan-like coloring when applied to human skin.

The term “long-term” or “long-lasting” as used herein refers to continued appearance of darkening or color after the initial application, even after washing the skin with a mild non-abrasive cleanser. In one embodiments, the darkening or color last for a few days. In another embodiment, the darkening or color lasts for a few weeks.

The term “patch” as used herein refers to a flexible planar surface which can be placed on skin. Patches can be made of any flexible material such as paper, woven material, porous material, semi-porous or partially-porous material, plastic films or metal films. Patches can be a single layer or multilayered, for example, having an absorbent layer and a waterproof layer. The patches can be in any number of shapes and sizes.

### Skin Darkening Agents

Skin darkening agents use a variety of different methods to darken skin. Some deposit color on the surface of the skin, others dye the skin and yet others cause an increase in pigmentation in the skin. The lasting effects of the skin darkening agents also vary. Some wash off with soap and water while others last of several days or even weeks. Non-limiting examples of skin darkening agents include dihydroxyacetone (CAS number 96-26-4), erythrulose (CAS number 533-50-6), henna, glucose tyrosinate, acetyl tyrosine, brazilin, caffine, coffee extracts, isobutyl methyl xanthine, methyl xanthine, tea extracts, theophylline, isatin, alloxan, ninhydrin, glyceraldehyde, mesotartaric aldehyde, glutaraldehyde, pyrazoline-derivatives of 4,5-diones, derivatives of 4,4-dihydroxypyrazolin-5-ones, walnut extract (e.g. juglone), cacao extract, spices (e.g. saffron, curry, turmeric, cinnamon, cloves, allspice, nutmeg, paprika, rosemary, vanilla, cumin, etc.), fruit extract (e.g. grapes, tomatoes, etc.), vegetable extract (e.g. carrots, beets, cabbage, etc.), and berry extract (e.g. strawberries, blueberries, blackberries, mulberries, raspberries, etc.). In one embodiment, two or more skin darkening agents are used. In another embodiment, three or more skin darkening agents are used. In another embodiment, four or more skin darkening agents are used. In another embodiment, five or more skin darkening agents are used.

A. Dihydroxyacetone

Dihydroxyacetone (DHA) is a 3-carbon sugar involved in carbohydrate metabolism in higher plants and animals. It is a physiologic product of the body and is presumed to be nontoxic. DHA darkens the skin by binding to amines, peptides and free amino acids of the outer layers of the stratum corneum to generate a Maillard reaction. Different amino acids react to DHA in different ways, producing different tones of coloration from yellow to brown. The resulting pigments are called melanoidins.

DHA has been used in sunless tanning products since the 1960's. After contact with DHA, the skin begins to “tan” within two or three hours and continues to darken for approximately six hours. The result is a substantive tan that lasts until the outer layers of the skin slough off. The tan's intensity depends on the type and thickness of the stratum corneum of the skin. Areas where the stratum corneum is thick (e.g. elbows) result in a more intense tan and areas where the stratum corneum is thin (e.g. face) the tan is less intense. One of the drawbacks of DHA is its propensity to impart an orange color on the skin. This can be corrected with the addition of dyes and other additives to create a more natural tan color.

B. Erythrulose

Erythrulose is a natural keto-sugar occurring in Red Raspberries, and may also be produced by fermentation of the bacterium Gluconobacter followed by multiple purification steps. Erythrulose reacts with free primary or second amino groups of keratin in the upper layers of the epidermis. This conversion of reducing sugar with amino acids, peptides or proteins, similar to the “Maillard reaction”, also known as non-enzymatic browning, leads to the formation of brownish polymers, the so-called melanoids. The resulting brown polymers are bound to proteins of the stratum corneum mainly via lysine side-chains. The brown color is comparable to the appearance of natural sun tan. Tanning effect appears in 2-3 days, the maximal tanning intensity is reached with Erythrulose after 4 to 6 days. The tanned appearance typically lasts from 2 to 10 days depending on application type, and skin condition.

C. Henna

Henna is a dye extracted from the Lawsonia inermis plant and often used in temporary tattoos. Henna has been used since antiquity to dye skin, hair, and fingernails, as well as fabrics including silk, wool, and leather. The active compound of henna is a molecule called lawsone, which binds to proteins in the skin and imparts a red-brown color. The leaves of the Lawsonia plant are mashed into a gel and applied to the skin. A light stain may be achieved within minutes, but the longer the paste is left on the skin, the darker and longer lasting the stain will be. Henna stains are orange when the paste is first removed, but darken over the following three days to a deep reddish brown. The stain lasts 10-20 days and fades as the skin exfoliates the stained skin cells.

While the use of “neutral henna” and “black henna” are contemplated, the use of henna is preferred. Neutral henna is usually the powder of the Senna italic plant. Black henna is usually derived from the indigo plant and may also contain other dyes and chemicals including p-phenylenediamine (PPD), which can stain skin black quickly, but can cause severe allergic reactions and permanent scarring.

### Device/Applicator

The long term skin darkening agents can be applied to the skin using various devices. Non-limiting devices include patches, pencils, sticks, sponges, sprayers, brushes, and compacts. The devices used will depend on the formulation of the skin darkening agent. Non-limiting formulations include, liquids, gels, solids, semi-solids, powders, and aerosols.

A. Patches

Patches are known in the cosmetics industry and have been used to treat acne, wrinkles, clogged pores, blemishes, and other cosmetic conditions. The patch may be made of any material that is flexible enough to conform to the contours of the body, in particular, the face. Non-limiting examples of patch material include filter paper, woven material, non-woven material, plastic films, and thin metals (e.g. foils). In one embodiment, the patch is a single layer. In another embodiment, the patch has at least two layers. In another embodiment, the patch has at least three layers. In another embodiment, the patch has at least four layers. In another embodiment, the patch has at least five layers.

In one embodiment, at least one layer contains a skin darkening agent. In one embodiment, one layer can be waterproof to protect clothing or other body parts. In one embodiment, one layer can be a non-permeable pouch that contains an accelerant or a retardant; the pouch containing the accelerant or retardant is broken upon application of the patch. In one embodiment, one layer can include a gel that heats or cools upon exposure to air or upon external pressure. In one embodiment, the outer most layer (i.e. the layer furthest from the skin) can be of a material that makeup will adhere to, allowing a user to disguise the patch.

In one embodiment, the patch has three layers, a backing film, an adhesive layer, and a release liner. The backing film is the layer that is furthest from the skin after application. The backing film protects the adhesive layer from external factors and provides characteristics as flexibility, occlusivity, breathability, and printability. Non-limiting examples of materials for the backing layer include, paper, non-woven material, plastic films such as polyethylene or polyester, polyurethane, or nylon. The release liner protects the product before the use of the product. The release liner is removed immediately prior to placement of the patch on the skin. Non-limiting examples of release liners include paper such as glassine paper, densified Kraft super-calendered paper, clay-coated paper, polyolefine coated paper; plastics such as polystyrene, polyester, polyethylene, case polypropylene, polyvinyl chlorice; and composite material based on the combination of several films. The release layer may optionally be coated with silicon or polyfluorocarbon. The adhesive layer contains an adhesive to make the patch stick to the skin as well as the active ingredient, e.g. skin darkening agent. Pressure sensitive adhesives stick to the skin with the application of very slight pressure. Non-limiting examples of pressure sensitive adhesives includes acrylic based, silicone based, and rubber based. An alternative to pressure sensitive adhesives are dry adhesive systems. In a dry adhesives system, the adhesive layer is created in situ by wetting the dry adhesive layer. Non-limiting examples of dry adhesives include synthetic or natural derivatives such as polyvinyl derivatives, starches, celluloses, and sugars. Each layer must be selected for its compatibility with the product.

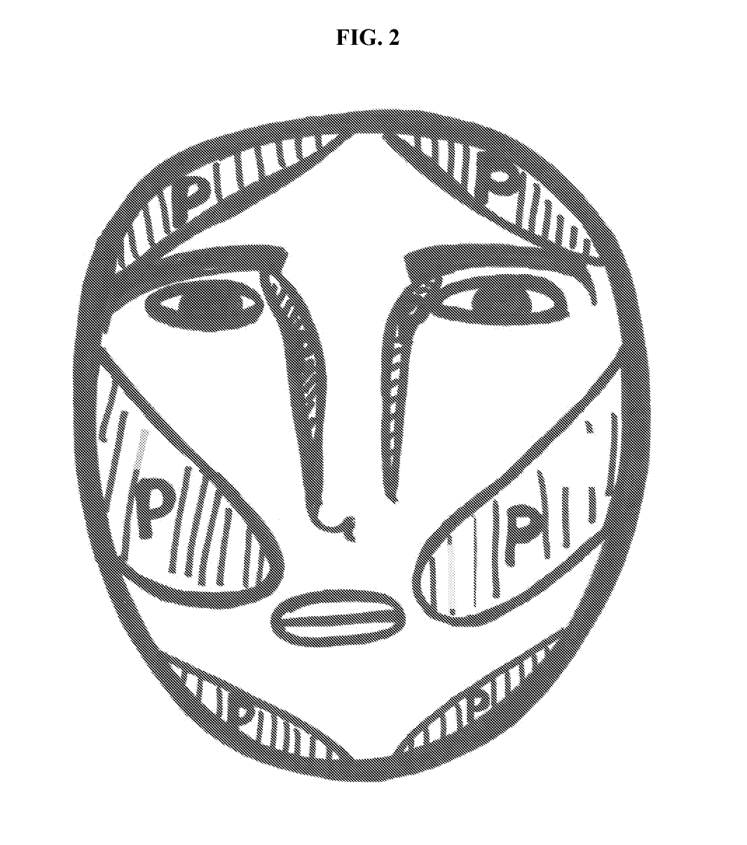

The shape of the patches may vary depending on where the patches are to be placed on the body. In one embodiment, the patches are shaped to mimic the application of the darker foundation in contour body and facial makeup. Non-limiting exemplary shapes are shown in FIG. 2. In another embodiment, the patches are basic shapes such as squares, rectangles, ovals, or circles. The basic shaped patches, in certain embodiments, can be cut by the user to any shape s/he desires. In another embodiment, referring to the layer patch described above, the backing layer may be noticeably larger than the adhesive layer. In a non-limiting example, the adhesive layer may be in the general shape of the number three “3” designed to be placed along the hairline, below the cheekbone, and along the jawline and the backing layer would be rectangular in shape.

The size of the patches can vary depending on where on the body the patch will be applied or the size of the features of the user. In one embodiment the patches are small enough in size to be applied to the side of a nose or along the crease of an eyelid. In another embodiment, the patches may cover a quarter of the face. In another embodiment, the patch may cover half of the face. In another embodiment, the patch may cover three quarters of the face. In another embodiment, the patch may cover the entire face. In another embodiment, the patch may cover at least a portion of the neck region. In another embodiment, the patch may cover at least a portion of the chest. In another embodiment, the patch may cover the at least a portion of stomach and/or abdomen. In another embodiment, the patch may cover at least a portion of the shoulders. In another embodiment, the patch may cover at least a portion of the upper arm. In another embodiment, the patch may cover at least a portion of the upper leg. In another embodiment, the patch may cover at least a portion of the lower leg. In another embodiment, the patch may cover at least a portion of the back. In another embodiment, the patch may cover at least a portion of the pelvic region.

The amount of the skin darkening agent may vary depending on the location on the adhesive layer. In a non-limiting example, the center of the adhesive layer may contain more skin darkening agent than the edges. This should result in a darkened region that is darkest in the center and fades out to the edges. This would create shadow areas that would be easier to blend into the contour makeup. For example, the center of the adhesive layer may contain a higher concentration of skin darkening agent that the edges. In one embodiment, the center of the adhesive layer may contain a 5% higher concentration, 10% higher concentration, 20% higher concentration, 30% higher concentration, 40% higher concentration, 50% higher concentration, 60% higher concentration, 70% higher concentration, 80% higher concentration, 90% higher concentration, 100% higher concentration, 125% higher concentration, 150% higher concentration, 175% higher concentration, 200% higher concentration, 250% higher concentration, 300% higher concentration, 350% higher concentration, 400% higher concentration, 450% higher concentration, 500% higher concentration, 600% higher concentration, 700% concentration, 800% concentration, 900% concentration, or 1000% higher concentration of the skin darkening agent than the edges. Alternatively, the skin darkening agent may only be applied to the center of the patch and allowed to bleed out to the edges of the patch. In another alternative, the center may contain more volume of the skin darkening agent than the edges. In one embodiment, the center of the adhesive layer may contain 5% more volume, 10% more volume, 15% more volume, 20% more volume, 25% more volume, 30% more volume, 35% more volume, 40% more volume, 45% more volume, 50% more volume, 55% more volume, 60% more volume, 65% more volume, 70% more volume, 75% more volume, 80% more volume, 85% more volume, 90% more volume, 95% more volume, or 100% more volume of the skin darkening agent than the edges.

B. Stick Shaped Device

In one embodiment, the device is stick shaped. The stick shaped device can be similar to a pencil, eye liner, or a tube of lipstick. The dye surface may be extended or retracted using a twisting or sliding motion. The stick shaped device is ideal for drawing discrete regions on the skin.

The stick shaped device may include makeup colors such that the initial application would provide the same affect as using makeup. Accordingly, once the makeup is removed, area of the skin would continue to remain darkened due to the skin darkening agent in the stick shaped device.

C. Sponges and Brushes

There are a variety of sponges and brushes that are available for applying makeup. The shapes and sizes vary depending on the use. Sponges and brushes come in a variety of shapes and sizes. The shapes can range from the tip of a pencil to a wide fan. For example, sponges for applying foundation are much larger than sponge tipped applicators for applying eye shadow. In one embodiment, the sponge or brush have hollow handles that can hold a skin darkening agent with or without makeup such as foundation. A mechanical feature would force the skin darkening agent (with or without makeup) to move from inside the handle to the sponge or brush so that it can then be applied to the skin. Non-limiting examples of the mechanical feature include external manual pressure, plunger, push button, twisting mechanism, or sliding mechanism. The sponges and brushes can be single use disposable devices or multi-use devices that can be cleaned and refilled.

D. Compacts

Also contemplated are compacts containing pressed powder. The pressed powder may be a mineral, dry powder, liquid powder, or paste. Compacts may include a brush, sponge, or pad to use to apply the contents of the compact. For dry powders, it is also contemplated to use a separate activating agent to activate the skin darkening agent if required. For example, DHA and erythrulose need to be in liquid form to work. The activating agent can be an aqueous based liquid or gel that is either mixed with the powder immediately before to applying on the skin or is applied to the skin after the powder is applied to the skin. Additionally, the compact may have a mirror.

### Formulations

The formulations are designed to (1) minimize side effects to the end user, (2) maximize the efficiency of the skin darkening agent, and (3) be easily applied to skin. The formulations are based on ingredients commonly used in the cosmetics industry to formulate liquids, sprays, lotions, gels, emulsions, creams, pastes, ointments, powders, and the like. Non-limiting examples of such ingredients can be found references such as “Cosmetic Formulations of Skin Care Products,” (ISBN-13: 978-0849339684, Draelos and Thaman, eds., CRC Press, 2005); “Chemistry and Manufacture of Cosmetics, Volume II Formulating,” (ISBN-13: 978-1932633481, Schlossman, ed., 4^{th} ed., Allured Pub Corp, 2009); and “Chemistry and Manufacture of Cosmetics, Volume III Ingredients,” (ISBN-13: 978-0931710773, Schlossman, ed., 3^{rd} ed., Allured Pub Corp 2002), the contents of which are incorporated herein.

For maximum stability, DHA is should be used in un-buffered acidic systems. The optium pH range is between 2 and 4. DHA can be combined with most cosmetic ingredients in all types of formulations with some limitations. DHA formulations should avoid amines such as aromatic amines, including PVP and tertiary amines, including EDTA as these can react with DHA. Other sensitive materials include acrylates or methacrylates, fruit acids (α-hydroxy acids), inorganic UV filters such as titanium dioxide and zinc oxide and phosphoric acid esters and their salts. Low pH is preferred as an alkaline pH tends to bring out orange colors. Other incompatible ingredients include phenoxyethanol, benzoic acid, dehydroacetic acid, potassium sorbate, sodium hydroxymethylglycinate, imidazolidinyl urea, carbomer, acrylates/c10-30 alkyl acrylate crosspolymer, polyacrylamide, c13-14 isoparaffin, laureth-7, zinc oxide, caprylic/capric triglyceride, water, titanium dioxide, and chitosan.

Similar to DHA, erythrulose should be used in an un-buffered acidic system with a pH lower than 5.5. The presence of nitrogen-containing molecules in a cosmetic formulation can trigger the reactivity of erythrulose as can aromatic amines, including PVP and tertiary amines such as EDTA. Although erythrulose is more stable and better compatible with cosmetic ingredients compared with DHA, it is still recommended that the incompatibility and precautions of DHA are taken into account in designing erythrulose-containing formulations.

The concentration of the skin darkening agent may vary depending on the level of darkness desired. In one embodiment, the concentration of skin darkening agent is up to 0.5%, 1%, 2%, 3%, 4%, 5%, 6%, 7%, 8%, 9%, 10%, 15%, 20%, 30%, 40%, or 50%. The formulation can also include accelerators to decrease the amount of time needed to achieve maximum darkening. Non-limiting examples of accelerators include troxerutin, rutin, rutin sulfate, propylene glycol, walnut extract, Lawsonia inermis extract, Eclipta prostrata extract, Eclipta alba Hassk extract, psoralens, acetyl tyrosine, tyrosine, and dimethyl isosorbide.

The formulation of the skin darkening agent will depend on the device/applicator being used. For example, gels or creams work best for patches whereas solids or semi-solids work best for the stick like devices. Non-limiting examples of formulations include, gels, creams, lotions, emulsions, powder, liquid powders, aerosols, spray, liquid, oil, ointment, or dry oil. Formulations for patches may need to take into account that some compounds may cause increased skin irritation when applied under occlusion. Accordingly, it may be beneficial to include skin soothing compounds to prevent or treat irritation and inflammation. Non-limiting examples of skin soothing compounds include anti-inflammatory, moisturizers, antioxidants, strontium, oat extract, soy extracts, feverfew, and chamomile. Compounds applied to the skin under occlusion may also be absorbed at a much higher rate due to the highly hydrated state of the skin. The concentration of the skin darkening agent may need to be adjusted accordingly.

The skin darkening agent may be combined with makeup. In one embodiment, the makeup is the type used for contour makeup. In another embodiment, the makeup is the type used for the darkening regions of contour makeup to create shadow areas. Non-limiting examples include foundation, press powder, or bronzers.

Also contemplated are additional compounds that could make the skin darkening effects last longer. Non-limiting examples include skin penetration enhancers (e.g. phospholipids (e.g. empty liposomes), lecithin, glycerin/glycerol, disodium phosphate, ethoxydiglycol, dimethyl isosorbide, alpha bisobola), dye stabilizers, moisturizers, oils, etc. Regarding the use of skin penetrations enhancers, it is understood that the skin darkening agent should remain within the epidermis to produce the visual affect. Additionally, compounds that accelerate the skin darkening effects may be included. Non-limiting examples include phospholipids with unsaturated side chain (e.g. phosphatidyl choline, lecithin, “empty” liposomes), troxerutin, propylene glycol, walnut extract, henna, eclipta prostrate extract, tyrosine, agents that increase blood circulation (e.g. menthol, benzyl nicotinate, methyl nicotinate, hexyl nicotinate, ethylene glycol, camphor, etc.), etc.

Additional ingredients known in the cosmetics art may be included. Non-limiting examples include those that may contribute to improving the skin (e.g. moisturizers, anti-acne compounds, anti-irritant compounds, sunscreen, vitamins, minerals, anti-aging compounds, anti-wrinkle compounds, skin lightening compounds, etc.); the look and feel of the product (e.g. colorants, pigments, fillers, polymers, fragrances, dyes, clay, absorbent powders, surfactants, solvents, gelling agent, waxes, oils, silicon, light scattering compounds, etc.); extend shelf-life (e.g. preservatives (e.g. benzoic acid, sodium benzoate, methylparaben, ethylparaben, butylparaben, propylparaben, benzyalkonium chloride, phenylmercuric nitrate, and chlorhexidine), antioxidants, metal chelator (e.g. ethylenediaminetetraacetic acid, citric acid, phytic acid, ethylenediamine, porphine, dimercaprol, etc.), stabilizers, etc.); or prevent the growth of microorganisms (e.g. parabens, etc.).

### Methods of Use

The skin darkening agents of the present disclosure are designed to be applied to the skin to visually create shadow areas associated with contour makeup. The amount of skin darkening achieved depends on many factors including skin tone, concentration of the skin darkening agent, and amount of time the skin darkening agent is left on the skin.

In one embodiment, the skin darkening agent is part of a patch. The patch is applied to the skin and left on for an amount of time. In one embodiment, the patch is designed to be left on for up to 5 minutes, 10 minutes, 15 minutes, 20 minutes, 25 minutes, 30 minutes, 35, minutes, 40 minutes, 45 minutes, 50 minutes, 55 minutes, 60 minutes, 2 hours, 3 hours, 4 hours, 5 hours, 6 hours, 7 hours, 8 hours, 9 hours, 10 hours, 11 hours, 12 hours, 13 hours, 14, hours, 15 hours, 16 hours, 17 hours, 18 hours, 19 hours, 20 hours, 21 hours, 22 hours, 23 hours, or 24 hours. After the patch is removed, the area under the patch would begin and/or continue to darken over time.

In another embodiment, the skin darkening agent is combined with makeup products such as foundation used in contour makeup. The user applies the foundation to create the shadowed areas. The foundation provides the color where it is applied. Once the foundation is washed off, the skin would remain darker due to the skin darkening agent.

### Computer System

Also contemplated in this disclosure is the use of a computer system that would recommend placement of contour makeup based on an image that is uploaded, scanned in, or taken with a camera. In another embodiment, the person can choose a final makeup look. Non-limiting examples of final makeup looks include a natural look, daytime look, evening look, or dramatic look. In another embodiment, the system includes a connection to a database containing information on skin type, facial structure, eye shape, nose shape, lip shape, skin darkening agents and recommended exposure time based on concentration and chemical, and various makeup looks. In one embodiment, the computer system is a smart phone app that uses the camera phone to obtain an image of a person's face. In another embodiment, the computer system is connected to a printer that can custom design patch shapes for a particular person.

### EXAMPLES

### Example 1

### Skin Darkening Based on Exposure Time to DHA

In this example, strips of filter paper are soaked in a solution containing a 5% concentration of DHA. The skin on the underside of the forearm is cleaned and exfoliated. The filter paper is applied to the forearm for 5 minutes, 10 minutes, 15 minutes, 20 minutes, 30 minutes, or 1 hour. The strips are removed and the skin is patted dry. The skin is evaluated for the darkening effect after 1 hour, 3 hours, 6 hours, 10 hours, and 24 hours. The skin is also evaluated after 3 days, 5 days, 7 days, 10 days, 12 days, and 15 days to determine how long the tan lasts.

### Example 2

### Skin Darkening Based on Concentration of DHA

In this example, strips of filter paper are soaked in a solution containing a 1%, 2%, 5%, or 10% concentration of DHA. The skin on the underside of the forearm is cleaned and exfoliated. The filter paper is applied to the forearm for a set 15 minutes. The strips are removed and the skin is patted dry. The skin is evaluated for the darkening effect after 1 hour, 3 hours, 6 hours, 10 hours, and 24 hours. The skin is also evaluated after 3 days, 5 days, 7 days, 10 days, 12 days, and 15 days to determine how long the tan lasts.

### Example 3

### Skin Darkening Using Two Different Concentrations of DHA

In this example, two different concentrations of DHA are applied to a circular piece of filter paper. A solution containing a 10% concentration of DHA is applied to the center of the paper and a solution containing a 5% concentration of DHA is applied to the perimeter of the paper. The skin on the underside of the forearm is cleaned and exfoliated. The filter paper is applied to the forearm for a set 15 minutes. The strips are removed and the skin is patted dry. The skin is evaluated for the darkening effect after 1 hour, 3 hours, 6 hours, 10 hours, and 24 hours. The skin is also evaluated after 3 days, 5 days, 7 days, 10 days, 12 days, and 15 days to determine how long the tan lasts.

### Example 4

### Skin Darkening Based on Exposure Time to Erythrulose

In this example, strips of filter paper are soaked in a solution containing a 5% concentration of erythrulose. The skin on the underside of the forearm is cleaned and exfoliated. The filter paper is applied to the forearm for 5 minutes, 10 minutes, 15 minutes, 20 minutes, 30 minutes, or 1 hour. The strips are removed and the skin is patted dry. The skin is evaluated for the darkening effect after 1 hour, 3 hours, 6 hours, 10 hours, and 24 hours. The skin is also evaluated after 3 days, 5 days, 7 days, 10 days, 12 days, and 15 days to determine how long the tan lasts.

### Example 5

### Skin Darkening Based on Concentration of Erythrulose

In this example, strips of filter paper are soaked in a solution containing a 1%, 2%, 5%, or 10% concentration of erythrulose. The skin on the underside of the forearm is cleaned and exfoliated. The filter paper is applied to the forearm for a set 15 minutes. The strips are removed and the skin is patted dry. The skin is evaluated for the darkening effect after 1 hour, 3 hours, 6 hours, 10 hours, and 24 hours. The skin is also evaluated after 3 days, 5 days, 7 days, 10 days, 12 days, and 15 days to determine how long the tan lasts.

### Example 6

### Skin Darkening Using Two Different Concentrations of Erythrulose

In this example, two different concentrations of erythrulose are applied to a circular piece of filter paper. A solution containing a 10% concentration of erythrulose is applied to the center of the paper and a solution containing a 5% concentration of erythrulose is applied to the perimeter of the paper. The skin on the underside of the forearm is cleaned and exfoliated. The filter paper is applied to the forearm for a set 15 minutes. The strips are removed and the skin is patted dry. The skin is evaluated for the darkening effect after 1 hour, 3 hours, 6 hours, 10 hours, and 24 hours. The skin is also evaluated after 3 days, 5 days, 7 days, 10 days, 12 days, and 15 days to determine how long the tan lasts.

### Example 7

### Skin Darkening Based on Exposure Time to a Mixture of DHA and Erythrulose

In this example, strips of filter paper are soaked in a solution containing a 2.5% concentration of DHA and a 2.5% concentration of erythrulose. The skin on the underside of the forearm is cleaned and exfoliated. The filter paper is applied to the forearm for 5 minutes, 10 minutes, 15 minutes, 20 minutes, 30 minutes, or 1 hour. The strips are removed and the skin is patted dry. The skin is evaluated for the darkening effect after 1 hour, 3 hours, 6 hours, 10 hours, and 24 hours. The skin is also evaluated after 3 days, 5 days, 7 days, 10 days, 12 days, and 15 days to determine how long the tan lasts.

### Example 8

### Skin Darkening Based on Concentration of a Mixture of DHA and Erythrulose

In this example, strips of filter paper are soaked in a solution containing a 0.5%, 1%, 2.5%, or 5% concentration of DHA and a 0.5%, 1%, 2.5%, or 5% concentration of erythrulose. The skin on the underside of the forearm is cleaned and exfoliated. The filter paper is applied to the forearm for 15 minutes. The strips are removed and the skin is patted dry. The skin is evaluated for the darkening effect after 1 hour, 3 hours, 6 hours, 10 hours, and 24 hours. The skin is also evaluated after 3 days, 5 days, 7 days, 10 days, 12 days, and 15 days to determine how long the tan lasts.

### Example 9

### Skin Darkening Using Two Different Concentrations of a Mixture of DHA and Erythrulose

In this example, two different concentrations of erythrulose are applied to a circular piece of filter paper. A solution containing a 5% concentration of DHA and a 5% concentration of erythrulose is applied to the center of the paper and a solution containing a 2.5% concentration of DHA and a 2.5% concentration of erythrulose is applied to the perimeter of the paper. The skin on the underside of the forearm is cleaned and exfoliated. The filter paper is applied to the forearm for 15 minutes. The strips are removed and the skin is patted dry. The skin is evaluated for the darkening effect after 1 hour, 3 hours, 6 hours, 10 hours, and 24 hours. The skin is also evaluated after 3 days, 5 days, 7 days, 10 days, 12 days, and 15 days to determine how long the tan lasts.

### Example 10

### Skin Darkening Using Foundation Containing DHA

In this example, the skin darkening effect of foundation makeup containing DHA is evaluated on a female subject. DHA is added to liquid makeup foundation to a final concentration of 3%. The subject's face is washed, exfoliated, and moisturized. Contour makeup is applied to the subjects face using the DHA foundation to create the shadow regions. The subject wears the DHA foundation for 4 hours. After 4 hours, all makeup is removed from the subject's face using soap and water. The skin is evaluated for the darkening effect after 1 hour, 3 hours, 6 hours, 10 hours, and 24 hours. The skin is also evaluated after 3 days, 5 days, 7 days, 10 days, 12 days, and 15 days to determine how long the tan lasts.

### Example 11

### Skin Darkening Using Foundation Containing Erythrulose

In this example, the skin darkening effect of foundation makeup containing erythrulose is evaluated on a female subject. Erythrulose is added to liquid makeup foundation to a final concentration of 3%. The subject's face is washed, exfoliated, and moisturized. Contour makeup is applied to the subjects face using the Erythrulose foundation to create the shadow regions. The subject wears the Erythrulose foundation for 4 hours. After 4 hours, all makeup is removed from the subject's face using soap and water. The skin is evaluated for the darkening effect after 1 hour, 3 hours, 6 hours, 10 hours, and 24 hours. The skin is also evaluated after 3 days, 5 days, 7 days, 10 days, 12 days, and 15 days to determine how long the tan lasts.

### Example 12

### Skin Darkening Using Foundation Containing DHA and Erythrulose

In this example, the skin darkening effect of foundation makeup containing DHA and erythrulose is evaluated on a female subject. DHA and erythrulose are added to liquid makeup foundation to a final concentration of 1.5% each. The subject's face is washed, exfoliated, and moisturized. Contour makeup is applied to the subjects face using the DHA/erythrulose foundation to create the shadow regions. The subject wears the DHA/erythrulose foundation for 4 hours. After 4 hours, all makeup is removed from the subject's face using soap and water. The skin is evaluated for the darkening effect after 1 hour, 3 hours, 6 hours, 10 hours, and 24 hours. The skin is also evaluated after 3 days, 5 days, 7 days, 10 days, 12 days, and 15 days to determine how long the tan lasts.

### Example 13

### Longer Lasting Skin Darkening Compounds

In this example, the lasting effect of DHA and erythrulose combined with a skin penetration enhancer are evaluated. Strips of filter paper are soaked in one of the following solutions (1) 5% concentration of DHA; (2) 5% concentration of DHA plus 10% concentration of glycerol; (3) 5% concentration of DHA plus 10% concentration of olive oil; (4) 5% concentration of DHA plus 10% concentration of coconut oil; (5) 5% concentration of erythrulose; (6) 5% concentration of erythrulose plus 10% concentration of glycerol; (7) 5% concentration of erythrulose plus 10% concentration of olive oil; and (8) 5% concentration of erythrulose plus 10% concentration of coconut oil. The skin on the underside of the forearm is cleaned and exfoliated. The filter paper is applied to the forearm for 15 minutes. The strips are removed and the skin is patted dry. The skin is evaluated for the darkening effect after 1 hour, 3 hours, 6 hours, 10 hours, and 24 hours. The skin is also evaluated after daily for three weeks to determine how long the tan lasts.

### Example 14

### Skin Darkening Using Natural Extracts

In this example, the skin darkening effect of several natural extracts are evaluated. The natural extracts include vanilla, saffron, turmeric, cinnamon, cloves, curry, all spice, nutmeg, paprika, red wine, beets, carrots, tomatoes, grapes, strawberries, blueberries, blackberries, mulberries, cacao, tea, and coffee. The spices are mixed in water; the fruits, vegetables, and berries are mashed, cooked to make a concentrate, and strained; and concentrates of the cacao, tea, and coffee are made. Strips of filter paper are soaked in each of the extract solutions. The skin on the underside of the forearm is cleaned and exfoliated. The filter paper is applied to the forearm for 30 minutes. The strips are removed and the skin is patted dry. The skin is evaluated for the color and intensity of the stain from each extract. The skin is also evaluated after 1 day, 2 days, 3 days, 4 days, 5 days, and 7 days to determine how long the stain lasts.

### Example 15

### Exemplary Formulation

Formulation 1: 1-10% DHA and 0.1-5% xanthan gum.

Formulation 2: 1-10% erythrulose and 0.1-5% xanthan gum.

Formulation 3: 1-10% DHA, 1-10% glycerol, and 0.1-5% xanthan gum.

Formulation 4: 1-10% erythrulose, 1-10% glycerol, and 0.1-5% xanthan gum.

Formulation 5: 1-10% DHA, 0.1-5% phytic acid, 0.5-5% strontium, and 0.1-5% xanthan gum.

Formulation 6: 1-10% erythrulose, 0.1-5% phytic acid, 0.5-5% strontium, and 0.1-5% xanthan gum.

Formulation 7: 1-10% DHA, 1-10% glycerol, 0.1-5% phytic acid, 0.5-5% strontium, and 0.1-5% xanthan gum.

Formulation 8: 1-10% erythrulose, 1-10% glycerol, 0.1-5% phytic acid, 0.5-5% strontium, and 0.1-5% xanthan gum.

The examples set forth above are provided to give those of ordinary skill in the art a complete disclosure and description of how to make and use embodiments of the compositions, and are not intended to limit the scope of what the inventors regard as their invention. Modifications of the above-described modes (for carrying out the invention that are obvious to persons of skill in the art) are intended to be within the scope of the following claims. All publications, patents and patent applications cited in this specification are incorporated herein by reference as if each such publication, patent or patent application were specifically and individually indicated to be incorporated herein by reference.

### Additional Embodiments

Additional embodiments are described in the following numbered paragraphs.

1. A device for applying a skin darkening agent in a discrete location to accentuate body contours comprising an applicator containing a skin darkening agent.

2. The device of claim 1, wherein the applicator is a patch.

3. The device of claim 2, wherein the patch is pre-shaped to contours parts of a face.

4. The device of claim 3, wherein the patch is pre-shaped to contour a cheek.

5. The device of claim 3, wherein the patch is pre-shaped to contour a nose.

6. The device of claim 3, wherein the patch is pre-shaped to contour a forehead.

7. The device of claim 3, wherein the patch is pre-shaped to contour a jawline.

8. The device of claim 2, wherein the patch is pre-shaped to contour parts of a neck.

9. The device of claim 2, wherein the patch is pre-shaped to contour parts of a chest.

10. The device of claim 2, wherein the patch is pre-shaped to contour parts of an arm.

11. The device of claim 2, wherein the patch is pre-shaped to contour parts of a leg.

12. The device of claim 2, wherein the patch is pre-shaped to contour parts of an abdomen.

13. The device of any one of claims 2-12, wherein the patch includes an adhesive layer.

14. The device of claim 1, wherein the applicator is a stick like device.

15. The device of claim 1, wherein the applicator is sponge.

16. The device of claim 1, wherein the applicator is a brush.

17. The device of claim 15 or 16 further comprising a handle on the applicator.

18. The device of claim 17, wherein the handle is hollow.

19. The device of claim 18, wherein the hollow handle further comprises a mechanical means for extruding a composition located inside the hollow handle.

20. The device of claim 1, wherein the device is a compact and the applicator is either a brush, sponge, or pad.

21. The device of any one of claims 1-20, wherein the skin darkening agent is selected from a group consisting of: dihydroxyacetone, erythrulose, henna, glucose tyrosinate, acetyl tyrosine, brazilin, caffine, coffee extracts, isobutyl methyl xanthine, methyl xanthine, tea extracts, theophylline, isatin, alloxan, ninhydrin, glyceraldehyde, mesotartaric aldehyde, glutaraldehyde, pyrazoline-derivatives of 4,5-diones, derivatives of 4,4-dihydroxypyrazolin-5-ones, or walnut extract, cacao extract, spice extract, fruit extract, vegetable extract, and berry extract.

22. The device of claim 21, wherein the skin darkening agent is a mixture of at least two skin darkening agents.

23. The device of claim 21, wherein the skin darkening agent is a mixture of at least three skin darkening agents.

24. The device of claim 21, wherein the skin darkening agent is dihydroxyacetone.

25. The device of claim 21, wherein the skin darkening agent is erythrulose.

26. The device of claim 21, wherein the skin darkening agent is henna.

27. The device of claim 21, wherein the skin darkening agent is a spice extract.

28. The device of claim 21, wherein the skin darkening agent is a fruit extract.

29. The device of claim 21, wherein the skin darkening agent is a vegetable extract.

30. The device of claim 21, wherein the skin darkening agent is a berry extract.

31. A method for creating non-permanent long-term darkened regions on the skin comprising using the device of any one of claims 1-29 to apply a skin darkening agent to specific region of the skin.

32. The method of claim 31, wherein the specific region of the skin is part of a face.

33. The method of claim 31, wherein the specific region is a part of a neck.

34. The method of claim 31, wherein the specific region is part of a chest.

35. The method of claim 31, wherein the specific region is part of an abdomen.

36. The method of claim 31, wherein the specific region is part of an arm.

37. The method of claim 31, wherein the specific region is part of a leg.

38. The method of claim 31, wherein the specific region is part of a shoulder.

39. The method of claim 31, wherein the specific region is part of a back.

40. The method of claim 31, wherein the specific region is part of a pelvis.

41. A method for creating long-lasting contour makeup for a face comprising using the device of any one of claims 1-30 to apply a skin darkening agent to an area where a darker foundation would be applied for contour makeup.

42. The method of claim 41, wherein the area is the side of a nose.

43. The method of claim 41, wherein the area is under the cheek bone.

44. The method of claim 41, wherein the area is along the jaw.

45. The method of claim 41, wherein the area is along the hairline.

46. The method of claim 41, wherein the area is along the chin.

47. A computer system for recommending placement of contour makeup comprising obtaining an image of the face; analyzing the face shape; and providing the placement of highlighting makeup and shadowing makeup on the image of the face.

48. The computer system of claim 47, further comprising a camera to capture the image of the face.

49. The computer system of claim 47, further comprising a printer that will print patch shapes that are specific for the imaged face.

50. The computer system of claim 47, wherein the system is an app on an electronic device.

51. The computer system of claim 50, further comprising using a camera on the electronic device to provide the image of the face.

52. A composition for topical application comprising a skin darkening agent in a carrier.

53. The composition of claim 52, wherein the skin darkening agent is selected from a group consisting of: dihydroxyacetone, erythrulose, henna, glucose tyrosinate, acetyl tyrosine, brazilin, caffine, coffee extracts, isobutyl methyl xanthine, methyl xanthine, tea extracts, theophylline, isatin, alloxan, ninhydrin, glyceraldehyde, mesotartaric aldehyde, glutaraldehyde, pyrazoline-derivatives of 4,5-diones, derivatives of 4,4-dihydroxypyrazolin-5-ones, or walnut extract, cacao extract, spice extract, fruit extract, vegetable extract, and berry extract.

54. The composition of claim 53, wherein the skin darkening agent is a mixture of at least two skin darkening agents.

55. The composition of claim 53, wherein the skin darkening agent is a mixture of at least three skin darkening agents.

56. The composition of claim 53, wherein the skin darkening agent is dihydroxyacetone.

57. The composition of claim 53, wherein the skin darkening agent is erythrulose.

58. The composition of claim 53, wherein the skin darkening agent is henna.

59. The composition of claim 53, wherein the skin darkening agent is a spice extract.

60. The composition of claim 53, wherein the skin darkening agent is a fruit extract.

61. The composition of claim 53, wherein the skin darkening agent is a vegetable extract.

62. The composition of claim 53, wherein the skin darkening agent is a berry extract.

63. The composition of any one of claims 52-62, further comprising a chelating agent.

64. The composition of any one of claims 52-62, further comprising an anti-irritant agent.

65. The composition of any one of claims 52-62, further comprising a skin penetration enhancer.

66. The composition of any one of claims 52-62, further comprising a thickener.

67. The composition of any one of claims 52-62, further comprising a skin darkening accelerant.

All publications, patents and patent applications cited in this specification are incorporated herein by reference as if each such publication, patent or patent application were specifically and individually indicated to be incorporated herein by reference.

1. A device for applying a skin darkening agent at a discrete location on a skin surface to accentuate body contours comprising an applicator containing a skin darkening agent.

2. The device of claim 1, wherein the applicator is a patch.

3. The device of claim 1, wherein the applicator is a stick like device.

4. The device of claim 1, wherein the applicator is sponge.

5. The device of claim 1, wherein the applicator is a brush.

6. The device of claim 1 wherein the skin darkening agent is selected from a group consisting of: dihydroxyacetone, erythrulose, henna, glucose tyrosinate, acetyl tyrosine, brazilin, caffine, coffee extracts, isobutyl methyl xanthine, methyl xanthine, tea extracts, theophylline, isatin, alloxan, ninhydrin, glyceraldehyde, mesotartaric aldehyde, glutaraldehyde, pyrazoline-derivatives of 4,5-diones, derivatives of 4,4-dihydroxypyrazolin-5-ones, or walnut extract, cacao extract, spice extract, fruit extract, vegetable extract, and berry extract.

7. The device of claim 6, wherein the skin darkening agent is dihydroxyacetone.

8. The device of claim 6, wherein the skin darkening agent is erythrulose.

9. A composition for topical application comprising at least one skin darkening agent in a carrier.

10. The composition of claim 9, wherein the skin darkening agent is selected from a group consisting of: dihydroxyacetone, erythrulose, henna, glucose tyrosinate, acetyl tyrosine, brazilin, caffine, coffee extracts, isobutyl methyl xanthine, methyl xanthine, tea extracts, theophylline, isatin, alloxan, ninhydrin, glyceraldehyde, mesotartaric aldehyde, glutaraldehyde, pyrazoline-derivatives of 4,5-diones, derivatives of 4,4-dihydroxypyrazolin-5-ones, or walnut extract, cacao extract, spice extract, fruit extract, vegetable extract, and berry extract.

11. The composition of claim 10, wherein the skin darkening agent is dihydroxyacetone.

12. The composition of claim 10, wherein the skin darkening agent is erythrulose.

13. The composition of claim 10, wherein the skin darkening agent is a mixture of at least two skin darkening agents.

14. The composition of claim 9, further comprising a chelating agent.

15. The composition of claim 9, further comprising a skin penetration enhancer.

16. The composition of claim 9, further comprising a thickener.

17. The composition of claim 9, further comprising a skin darkening accelerant.

18. A method for creating at least one non-permanent long-term darkened region at a discrete location on a skin surface comprising the step of apply a composition comprising at least one skin darkening agent and a carrier to at least one location on the skin surface.

19. The method of claim 18, wherein the at least one location on the skin surface is on a face.

20. The method of claim 18, further comprising using a device for applying the composition.
